(19) United States
(12) Patent Application Publication
(10) Pub. No.: US 2024/0011964 A1
(43) Pub. Date: Jan. 11, 2024

(54) APPARATUS AND METHOD TO SEPARATE INDIVIDUAL HYDROCARBONS FROM A COMPOSITION OF MULTIPLE HYDROCARBONS IN ONE OR MORE MATRICES

(71) Applicant: ORONO SPECTRAL SOLUTIONS, INC., Hermon, ME (US)

(72) Inventors: Dean Smith, Dover-Foxcroft, ME (US); Muhammad Hossen, Hillsboro, OR (US); Justin Smith, Dover-Foxcroft, ME (US)

(73) Assignee: ORONO SPECTRAL SOLUTIONS, INC., Hermon, ME (US)

(21) Appl. No.: 18/251,974

(22) PCT Filed: Nov. 8, 2021
(86) PCT No.: PCT/US2021/058487

§ 371 Date: May 5, 2023

Related U.S. Application Data
(60) Provisional application No. 63/111,076, filed on Nov. 8, 2020.

(51) Int. Cl. G01N 33/18; G01N 1/40

(52) U.S. Cl. CPC G01N 33/1833; G01N 1/405; G01N 1/4055; G01N 2001/4061

(57) ABSTRACT

An apparatus and method for separating hydrocarbons from a matrix sample that includes one or more nonpolar hydrocarbons. The apparatus includes a first container, a second container and a connector connecting the first container and the second container together. The connector is arranged to enable the passage of the matrix sample back and forth between the first container and the second container and includes an analyte retainer that captures polar hydrocarbons while allowing nonpolar hydrocarbons to pass through. The portion of the sample including the hydrocarbons is dried and reconstituted. It can then be transferred to a device configured to enable analysis of the matrix sample for detection of the at least one or more nonpolar hydrocarbons.

### BACKGROUND OF THE INVENTION

### 1. Field of the Invention

The present invention relates to devices and techniques used to distinguish particular types of hydrocarbons from one another in a matrix that may be a fluid. More particularly, the present invention relates to systems and methods for capturing hydrocarbons on a test bed for subsequent analysis in a test device. More specifically, the present invention relates to devices and methods for determining particular hydrocarbons in a matrix, including polar and nonpolar hydrocarbons.

### 2. Description of the Prior Art

There is a range of situations in which it is desirable to determine the composition of a matrix. For purposes of describing the present invention, a matrix may be a fluid, such as a solvent including water, a mixture, a gaseous material, or a loose solid such as soil but not limited thereto. In particular, there are situations in which it is of interest to determine whether one or more hydrocarbons exist in the matrix. That interest includes being able to identify the specific hydrocarbon or hydrocarbons that exist in a sample. Such analyses are conducted for many samples and so there is a need for a fast, safe, accurate, precise, more environmentally friendly (“green”), and economical hydrocarbon measurement system and technique used to measure the hydrocarbon content of the matrix. An aspect of such systems and techniques that exist is that one or more solvents are used to separate components of the sample composition. It is desirable to minimize the use of solvents while providing an effective and reliable analysis platform.

Infrared absorption measurements have been the preferred basis of measurement of hydrocarbon existence in a matrix, such as a water sample in particular, for many years. Generally, these measurement techniques require first performing a liquid-liquid extraction to remove the hydrocarbon from the matrix. Those solvents that have been considered effective for performing hydrocarbon extraction also raise environmental, health, and safety concerns. The sensing and detection industry response to this challenge has been to introduce new methods and systems for effective hydrocarbon separation and detection in a sample.

As used herein, “hydrocarbon” means all molecules containing hydrogen and carbon; examples include aliphatic and aromatic molecules as well as carboxyl groups in carboxylic acids or ester groups. As used herein, “oil” means a mixture of aliphatic hydrocarbons with generally between seven and 40 carbons in the chain, aromatic species, and other hydrocarbons. It includes crude oil, refined oil, heating oil, and any other form of carbon-based oil. As used herein, “TPH” means Total Petroleum Hydrocarbons, generally including non-volatile aliphatic molecules of varying chemical structure with up to 40 carbons. As used herein, “Grease” refers to long chain hydrocarbon molecules containing carbonyls such as carboxylic acid and/or ester functional group or groups. As used herein, “TOG” means Total Oil and Grease; that is, the total of TPH and Grease. As used herein, “HEM” means Hexane Extractable Material.

The current US standard method approved by the Environmental Protection Agency (“EPA”) for measuring TOG and TPH in a water sample is to take a liter sample of the composition to be analyzed, add a substantial volume of hexane, in the range of 90 to 130 milliliters (mls) and sometimes more, for TOG, and 90 to 130 mls for TPH, shake the sample, and let the water and solvent phase separate. The solvent phase is dried, and the remainder is weighed, with that dried mass remainder identified as the TOG. Next, the nonpolar portion of the TOG is identified by reconstituting the dried solvent phase remainder with hexane, passing that reconstituted remainder through a silica gel matrix, or adding silica gel to the reconstituted solvent followed by stirring and filtration of the silica gel, heating the reconstituted remainder to remove the hexane solvent, and the dried reconstituted remainder is weighed. That dried reconstituted remainder is the nonpolar portion of the TOG composition from the sample.

The existing EPA-approved extraction method requires the use of a large quantity, on the order of 180 mls or more, of hexane solvent. It also requires the use of highly accurate balances to measure as accurately as possible the weight of the dried solvent phase TOG remainder that is the TOG content of the sample, and the dried reconstituted remainder that is the nonpolar portion of the TOG. The existing method produces substantial hexane off gassing. This method is also labor intensive, and the measurement process can take a long time. It must be ensured that there is no water present, and all the hexane is evaporated, as the presence of either will result in over-reporting the TOG content of the sample.

What is needed is a reliable system and method to enable the detection of TOG in a matrix sample, as well as any polar and nonpolar hydrocarbons in the sample. The system and method must be relatively easy to use, and it must minimize solvent usage. Moreover, the system and method must be cost effective and reduce the importance of using highly accurate balances.

### SUMMARY OF THE INVENTION

It is an object of the present invention to provide a reliable apparatus and method to enable the detection of TOG in a matrix sample, as well as any polar and nonpolar hydrocarbons in the sample. It is also an object of the invention to provide such an apparatus and method that is relatively easy to use and that minimizes solvent usage, particularly in comparison to the amount of solvent needed under existing EPA-approved extraction methods. It is further an object of the invention to provide such an apparatus and method that is cost effective and reduces the need to use highly accurate balances.

These and other objects are achieved with the present invention, which is a hydrocarbon separation apparatus capable of obtaining test specimens suitable for use in detecting hydrocarbons in a matrix sample, and a related method for using that apparatus. The apparatus includes a first container, a second container, and a connector between the first container and the second container. The separation apparatus can be used to transfer captured hydrocarbon material to a specimen extractor apparatus and then to an analyzer for the analysis of hydrocarbons capture from a fluid sample.

The first container is used to collect a sample of a matrix such as a fluid to be analyzed. The matrix may be water but not limited thereto. The connector is used to transfer contents of the first container into the second container. The connector includes means for capturing a portion of the contents of the first container. The second container is used to reconstitute nonpolar elements of the contents transferred through the connector. Alternatively, the nonpolar constituents can be transferred to an additional sample container where reconstitution steps can be carried out. The second container may then be coupled to the specimen extractor apparatus, which includes a membrane for retaining analyte thereto. Specifically, the extractor apparatus is used to capture on the membrane the nonpolar hydrocarbon of the second container. The extractor apparatus may then be coupled to an analyzer, which analyzer is configured to identify the nonpolar hydrocarbon or hydrocarbons retained on the membrane. The analyzer may be a spectrometer, a radiometer or other detection tool configured with analysis software to enable hydrocarbon determination.

The membrane used in the extractor apparatus is preferably the type that contains minimal or no amount of the analyte of interest or minimal amounts or zero chemical bonds similar to the chemical bonds in the analyte of interest, which bonds may interfere with the wavelength detection range or ranges of interest. If the membrane contains the analyte or chemical bonds similar to the analyte, it must be such that they can be accounted for in the analysis of the tested membrane. For the purpose of determining hydrocarbon content in water, for example, the membrane contains minimal or zero hydrocarbon bonds which interfere with the wavelength detection range or ranges of interest. Such a membrane can be used to determine the type of hydrocarbon molecule present, and thus can differentiate TPH from TOG, without any separate sample preparation. The ClearShot™ Extractor system available from Orono Spectral Solutions of Hermon, Maine, US is suitable for this application. The OSS ClearShot method measures TOG by liquid-liquid extraction in syringe. In this process, after separating TOG in the separating container, fluid is removed, and solvent is evaporated. The remaining material is reconstituted using a solvent and that sample is processed through the extractor. In this case, the extracted TOG will not be passed through the connector to the second container.

The apparatus described is used to carry out the method for isolating TOG from the matrix, which may be a fluid such as water, and isolating nonpolar hydrocarbons of the TOG. The method includes the steps of directing a sample of the fluid to be analyzed into the first container and adding a small amount of hexane to the fluid in the first container. The combination is mixed and allowed to separate before removing the water phase from the first container to leave a hexane-based non-aqueous fluid in the first container. The connector includes a first end and a second end. The first end is removably connected to the first container and the second end is removably connected to the second container. The connector is a conduit with an interior through which fluid passes. The interior of the connector includes a selective fluid retainer chosen to capture within the connector at least a substantial portion of the polar portion of the TOG. That retainer may be an adsorbent selected to adsorb expected hydrocarbon components of the matrix under analysis. The retainer may be held in the connector with a mesh containment, for example. The adsorbent may be a powdered magnesium silicate or Silica Gel but not limited thereto.

The method further includes the steps of directing solvent including the analyte to be detected from the first container through the connector to the second container and capturing polar hydrocarbons of the non-aqueous fluid in the connector. The solvent and non-polar hydrocarbon fluid that passes into the second container can be heated and/or exposed to an air flow sufficient to cause evaporation of the solvent. The remaining dried non-polar hydrocarbon content is then reconstituted, such as with Acetone, Methanol, Ethanol, water, or other suitable solvent, and then may be transferred to the extractor apparatus. The fluid may be passed through the membrane of the extractor apparatus, which is selected to retain thereon the non-polar hydrocarbon. The membrane may then be placed in the analyzer for non-polar hydrocarbon analysis.

The apparatus and method of the present invention significantly reduce the use of solvent for effective extraction by as much as 99% in comparison to existing extractor methods. It eliminates the need to use very accurate balances as the nonpolar hydrocarbon specimen goes directly to the membrane and then directly to the analyzer. All sample processing can be carried out in the containers and connector, all of which may be enclosed, thereby reducing off gassing concerns. That closed system established with the apparatus is used to push the solvent such as hexane portion from one end of the apparatus to the other, thereby reducing solvent transfer steps. The result is an apparatus and method that can be used to identify specific oils in a sample and in the nonpolar portion of the sample as well. These and other features and advantages of the present invention will become apparent upon review of the following detailed description, the accompanying drawings and the appended claims.

### BRIEF DESCRIPTION OF THE DRAWINGS

FIG. 1 is a side view of primary components of the hydrocarbons separation apparatus of the present invention.

FIG. 2 is a side view of the first container of the apparatus showing wetting of the interior of the first container.

FIG. 3, including FIGS. 3A and 3B, shows the first container with sample dilution in FIG. 3A and liquid phase separation in FIG. 3B.

FIG. 4, including FIGS. 4A and 4B, shows the first container with attached plunger in FIG. 4A and being shaken in FIG. 4B.

FIG. 5, including FIGS. 5A and 5B, shows the first container with Luer cap in a shaker in FIG. 5A, and with solvent and aqueous layers separated after shaking in FIG. 5B.

FIG. 6, including FIGS. 6A and 6B, shows the first container with the aqueous layer being dispensed from the container in FIG. 6A and use of a needle through the Luer cap to remove residual aqueous layer from the hexane layer within the first container in FIG. 6B.

FIG. 7, including FIGS. 7A, 7B and 7C, shows the combination of the connector and second container being attached to the first container in FIG. 7A, transferring the solvent layer back and forth between the first container and the second container through the connector in FIG. 7B, and adding additional solvent to the solvent layer after final transfer into the second container in FIG. 7C.

FIG. 8, including FIGS. 8A, 8B, 8C and 8D, shows the second container detached from the combination of the first container and the connector in FIG. 8A, adding a Luer cap to the second container in FIG. 8B, removing the plunger from the second container in FIG. 8C, and heating the second container to remove solvent from the second container in FIG. 8D.

FIG. 9, including FIGS. 9A, 9B and 9C, shows the reconstitution of the sample in the second container after solvent removal by adding Acetone in FIG. 9B and water in FIG. 9B, and returning the plunger to the second container in FIG. 9C.

FIG. 10, including FIGS. 10A and 10B, shows shaking of the second container with its contents in FIG. 10A, and attachment of the extractor apparatus with membrane in FIG. 10B.

FIG. 11, including FIGS. 11A, 11B, and 11C, shows and describes elements of a first step associated with a second method for hydrocarbon separation of the present invention.

FIG. 12, including FIGS. 12A, 12B, 12C, 12D, and 12E, shows and describes elements of a second step associated with the second method.

FIG. 13, including FIGS. 13A, 13B, 13C, and 13D, shows and describes elements of a third step associated with the second method.

FIG. 14, including FIGS. 14A, 14B, 14C, 14D, and 14E, shows and describes elements of a fourth step associated with the second method.

FIG. 15, including FIGS. 15A and 15B, shows and describes elements of a fifth step associated with the second method.

FIG. 16, including FIGS. 16A, 16B, 16C, and 16D, shows and describes elements of a sixth step associated with the second method.

FIG. 17 shows and describes a seventh step associated with the second method.

FIG. 18, including FIGS. 18A, 18B, 18C, 18D, and 18E, shows and describes elements of an eighth step associated with the second method.

FIG. 19, including FIGS. 19A and 19B, shows and describes elements of a ninth step associated with the second method.

FIG. 20, including FIGS. 20A, 20B, 20C, and 20D, shows final analysis steps for either of the first and second methods described and illustrated.

### DETAILED DESCRIPTION OF THE INVENTION

In general, the present invention relates to the determination of analytes in fluids and, more particularly, to the determination of hydrocarbons in a matrix such as a fluid. Hydrocarbons in a matrix such as water are known to be harmful to the environment and human health. ‘Water’ can indicate fresh water, sea water, municipal wastewater, petroleum industry produced water (as used herein, “produced water” means wastewater produced, for example, in crude oil pumping or during industrial processing), bilge water from ships, and other waters. Each source of water has a limit to the concentration of hydrocarbons that can be present before the water can be discharged to the environment. Regulatory agencies worldwide enforce these limits by requiring periodic testing at industrial sites and others where hydrocarbons may be present in the water. The present invention seeks to provide an accurate, economical, rapid, environmentally-friendly solution to the problem of measuring hydrocarbons in water.

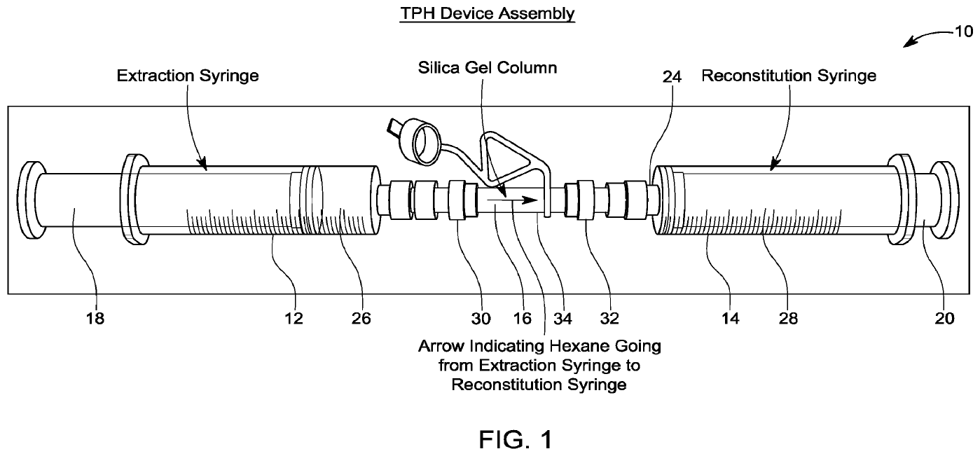

As illustrated in FIG. 1, a hydrocarbon separation apparatus 10 of the present invention includes a first container 12, a second container 14, and a connector 16. The first container 12 and the second container 14 may each be glass syringes or other material suitable for functioning as set out herein while in contact with the materials described. Each of the first container 12 and the second container 14 includes a removable plunger 18 and 20. The plungers 18 and 20 are used to draw fluid through inlet ports 22 and 24 into interior 26 and 28 of the containers 12 and 14, and to transfer fluid out of those interiors 26 and 28. The first container 12 is identified in the drawing as an extraction syringe and the second container 14 is identified in the drawing as a reconstitution syringe. It is noted that the functions of the first container 12 and second container 14 as described herein may be reversed such that the first container 12 may function as the reconstitution syringe and the second container 14 may function as the extraction syringe.

The connector 16 may be a non-hydrocarbon based and pressure-tight tube. It may be fabricated of a material suitable to continue its intended function while in contact with a matrix sample. For example, the connector 16 may be a metallic tube, a nonmetallic tube such as a plastic tube arranged or other type of material suitable to enable the passage of the matrix such as a fluid between the first container 12 and the second container 14 in either direction. The connector 16 may be chosen to select at least 50 psi of internal pressure. The connector 16 includes a first end 30 and a second end 32, wherein the first end 30 is removably connectable to the inlet port 22 of the first container 12 and the second end 32 is removably connectable to the inlet port 24 of the second container 14. The ends 30 and 32 of the connector 16 may be reversibly connected to the inlet 24 of the second container 14 and to the inlet 22 of the first container 12. The connector 16 includes an interior 36 through which fluid passes. In addition, when the apparatus 10 is in use for the purpose of separating hydrocarbons in a fluid, the interior 36 of the connector 16 is used to retain there in a hydrocarbon capture element 38, which may be Silica gel.

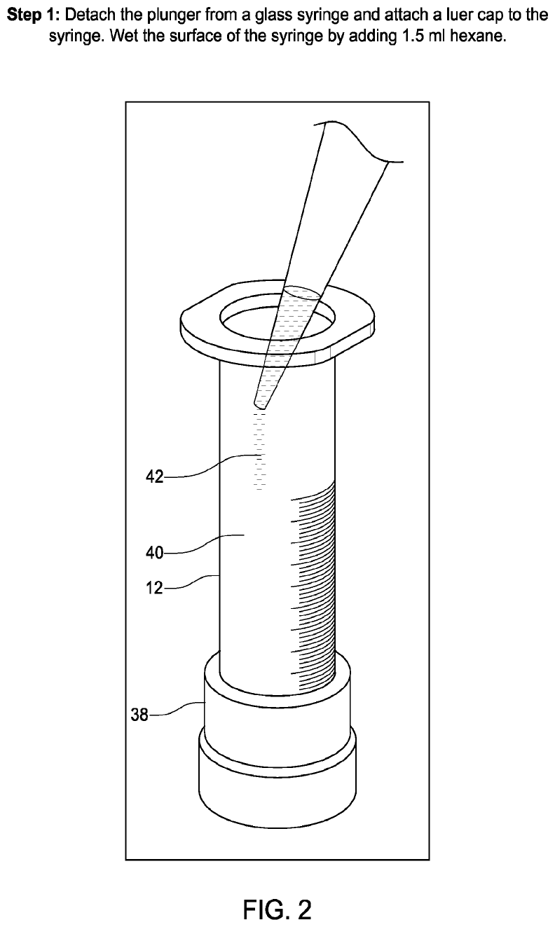

The apparatus 10 is used to perform steps of the method of the present invention to separate hydrocarbons in a matrix sample from one another for the purpose of subsequent use in an analyte analysis process. As illustrated in FIG. 2, a first step of the method includes detaching the plunger 18 of the first container 12 from the first container 12 and attaching a first Luer cap 38 to the inlet 22 of the first container 12. An interior surface 40 of the first container 12 is then wetted with a solvent 42. The amount of solvent 42 inserted into the first container 12 is minimal when compared to the volume of solvent used in prior separation actions. For example, the amount of wetting solvent 42 inserted is only about 1-3 mls. The solvent 42 may be hexane, pentane, or other solvent material suitable for rendering the analytes under review soluble.

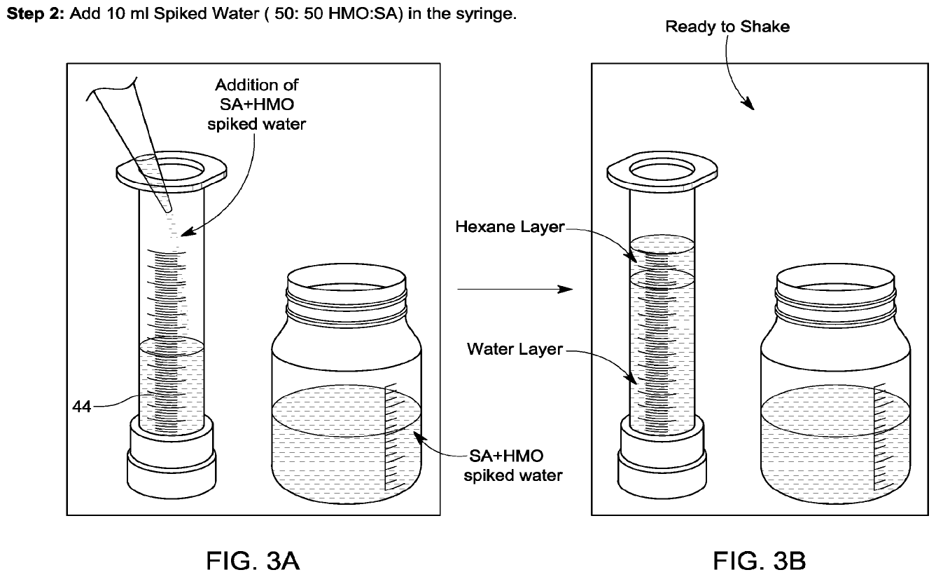

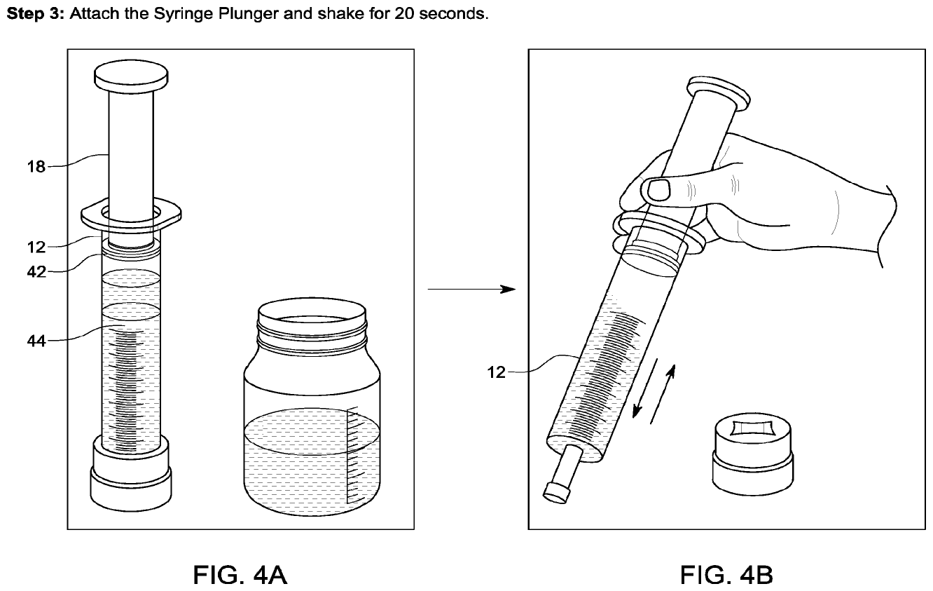

As illustrated in FIG. 3A, a sample 44 is inserted into the first container 12. The amount of sample 44 added is selectable and may be about 10 times the volume of wetting solvent inserted. For the purpose of illustrating an example of the function of the apparatus 10 only, the sample 44 is primarily water spiked with a 50/50 mixture of Heavy Mineral Oil (HMO)/Hexadecane and Stearic Acid (SA). Other hydrocarbon containing matrices such as fluids other than water can be subjected to separation using the apparatus 10. FIG. 3B shows the fluid solvent 42 and the sample 44 together and separated. FIG. 4A shows reattachment of the plunger 18 to the first container 12 for the purpose of sealing the combination of solvent 42 and sample 44 within the first container 12 for mixing. FIG. 4B shows the fluid in the first container 12 being shaken to form a mixture of the solvent 42 and the sample 44.

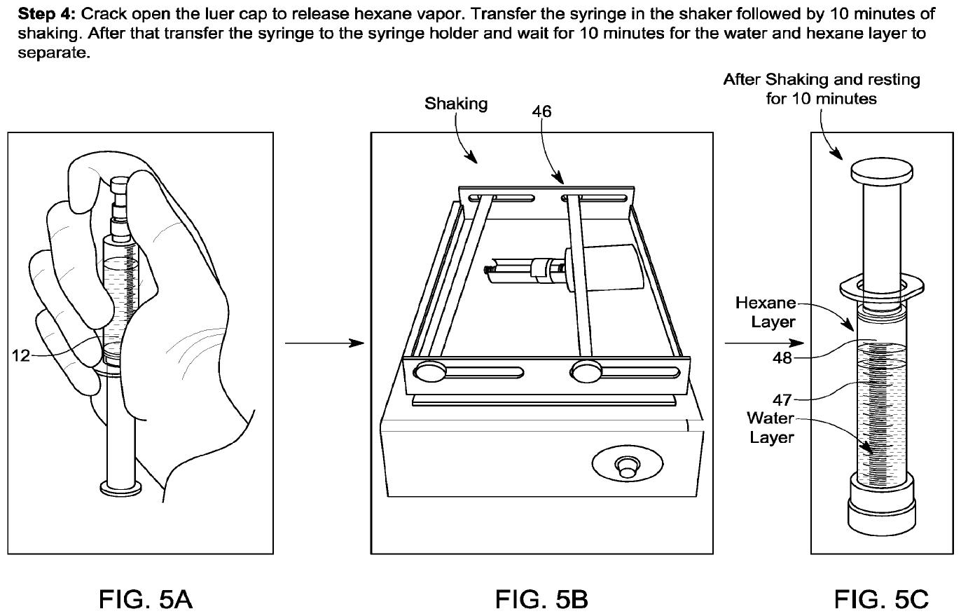

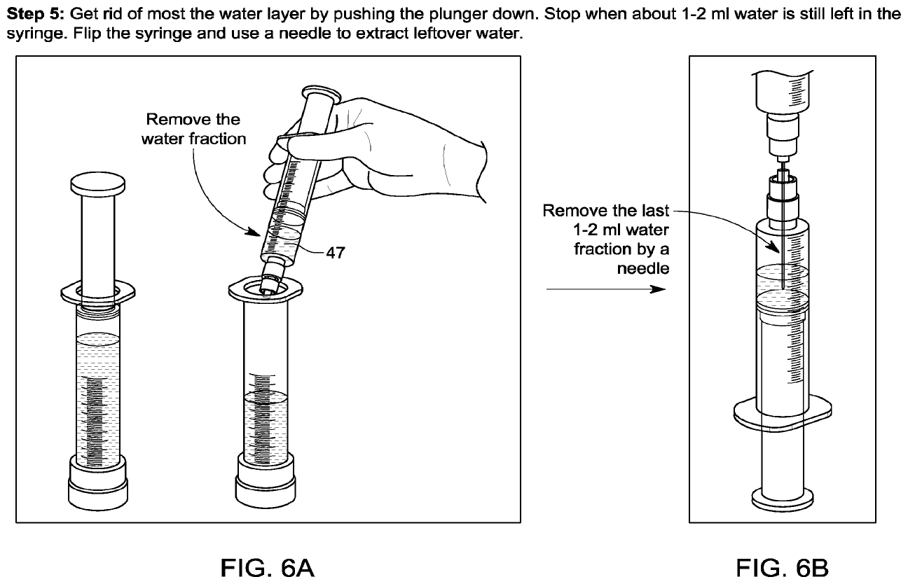

FIG. 5A shows the first container 12 Luer cap being opened. FIG. 5B shows the first container 12 in a shaking device 46 where the mixture is further shaken. This shaking causes hydrocarbons contained in the sample 44 to become soluble in the solvent 42 and thereby transferred from the sample 44 into the solvent 42 component of the mixture. FIG. 5C shows the mixture sample fluid layer 47 separated from hydrocarbon-containing solvent layer 48. As shown in FIG. 6A, the bulk of fluid layer 47 is removed from the interior 22 of the first container 12 after removal of the plunger 18, with any remainder removed with a needle, for example, as shown in FIG. 6B.

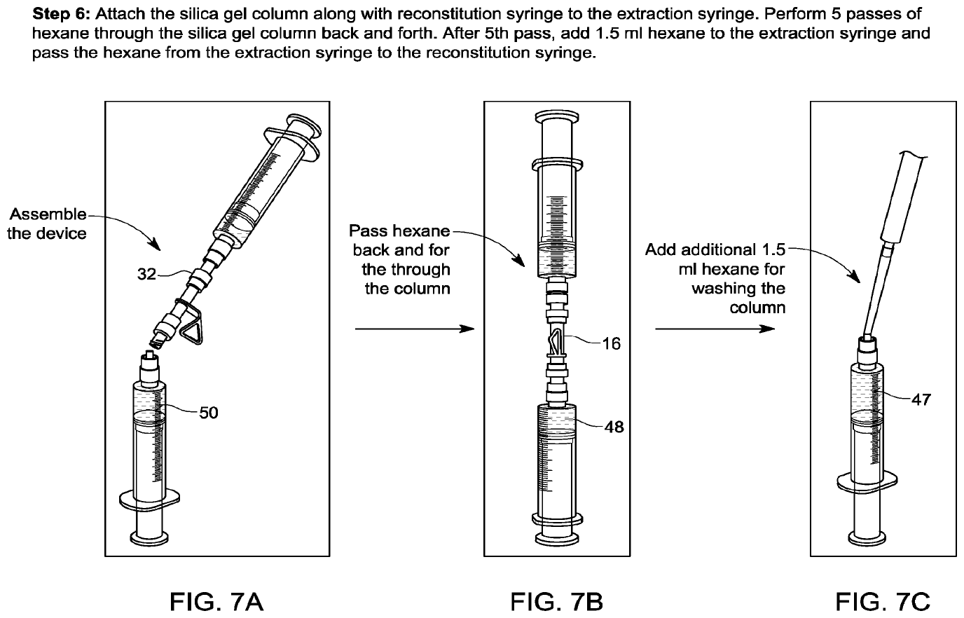

As shown in FIG. 7A, the second end 32 of the connector 16 is attached to second Luer cap 50 of the second container 14, and then the first end 30 of the connector 16 is attached to the first Luer cap 38 of the first container 12. The connector 16 includes Silica gel therein. The plungers 18 and 20 are also inserted into the first container 12 and the second container 14, respectively. As represented in FIG. 7B, the solvent layer 48 is transferred back and forth between the first container 12 and the second container 14 through the Silica gel of the connector 16 by coordinated actuation of the plungers 18 and 20. The transfer should be done a plurality of times. For example, five passes of the solvent layer 48 through the Silica gel, but not limited to that number of passes. The passing of the solvent layer 48 through the Silica gel traps polar hydrocarbons while allowing nonpolar hydrocarbons to pass through. As shown in FIG. 7C, upon completion of those passes, the first Luer cap 38 is removed from the first container 12 and a solvent wash is inserted into the first container 12. The first container 12, the second container 14 and the connector 16 are then reconnected and the solvent wash is transferred from the first container (extraction syringe) 12 through the connector 16 to the second container (reconstitution syringe) 14.

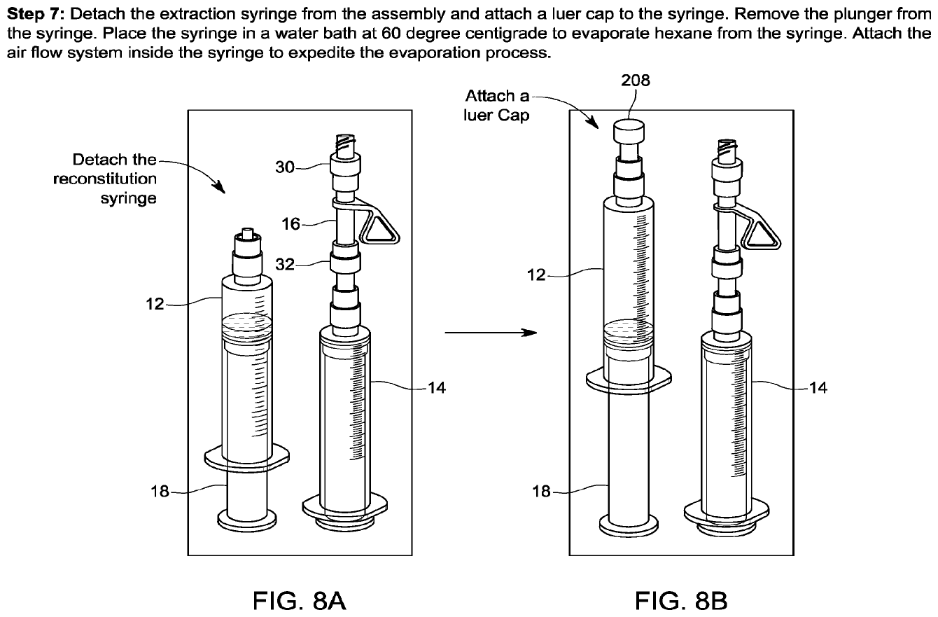

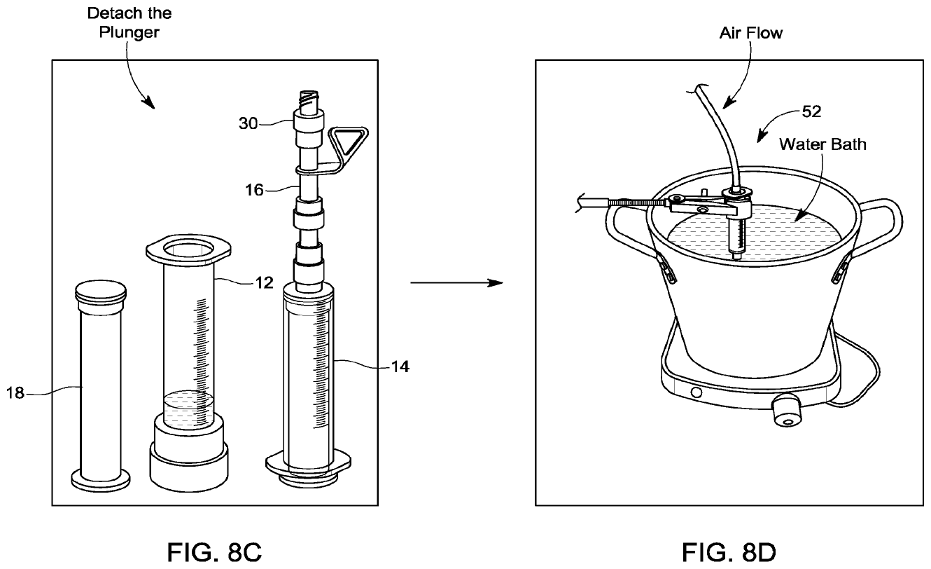

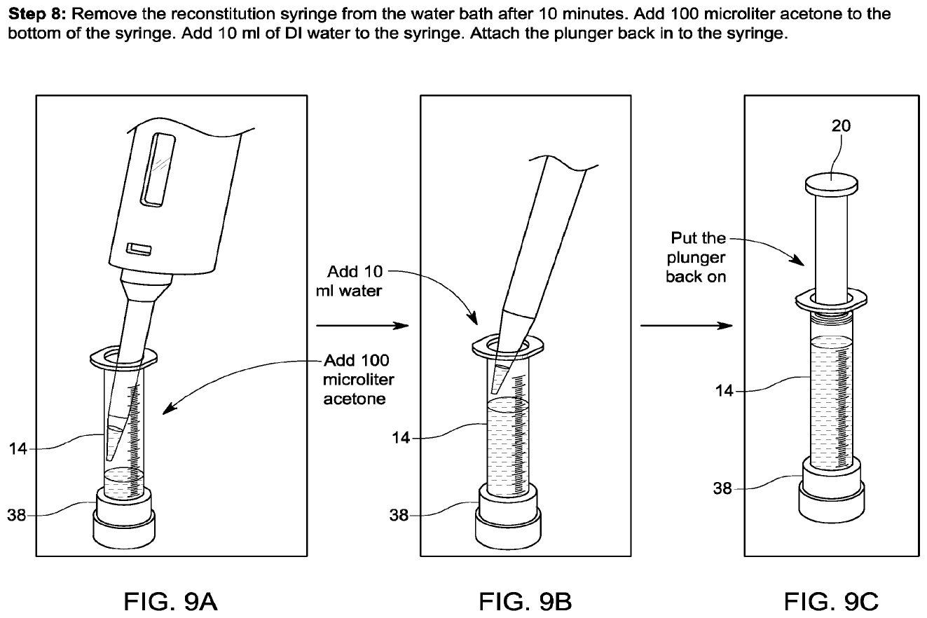

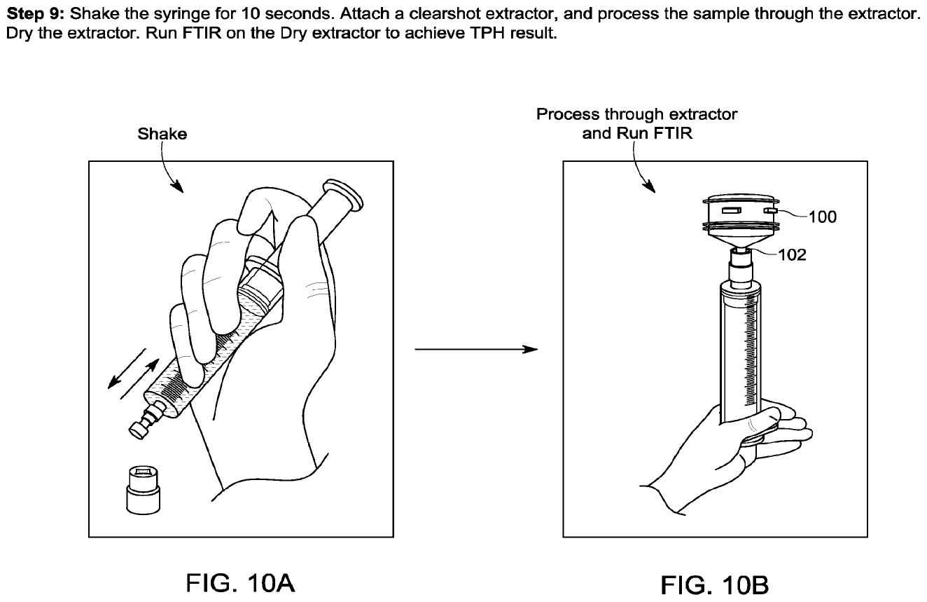

With reference to FIGS. 8A and 8B, the second container 14 containing the solvent layer 48 and solvent wash is disconnected from the connector 16, and the second Luer cap 50 applied to inlet port 24. The plunger 20 is removed from the second container 14 as shown in FIG. 8C, and then the open-ended second container 14 is inserted into a heating apparatus such as heating bath 52 to cause evaporation of solvent in the second container 16 as shown in FIG. 8D. As shown in FIGS. 9A to 9C, Acetone is added to the remainder in the second container 16 after cooling, water is also added to form a mixture containing one or more nonpolar hydrocarbons, and then the plunger 20 is reapplied to the second container 14. Finally, as shown in FIGS. 10A and 10B, the sealed second container 14 is shaken and then connected through the second Luer cap 50 to an extractor apparatus 100, which may include a membrane 102 for transfer of the mixture containing the one or more nonpolar hydrocarbons thereto for subsequent retained hydrocarbon analysis.

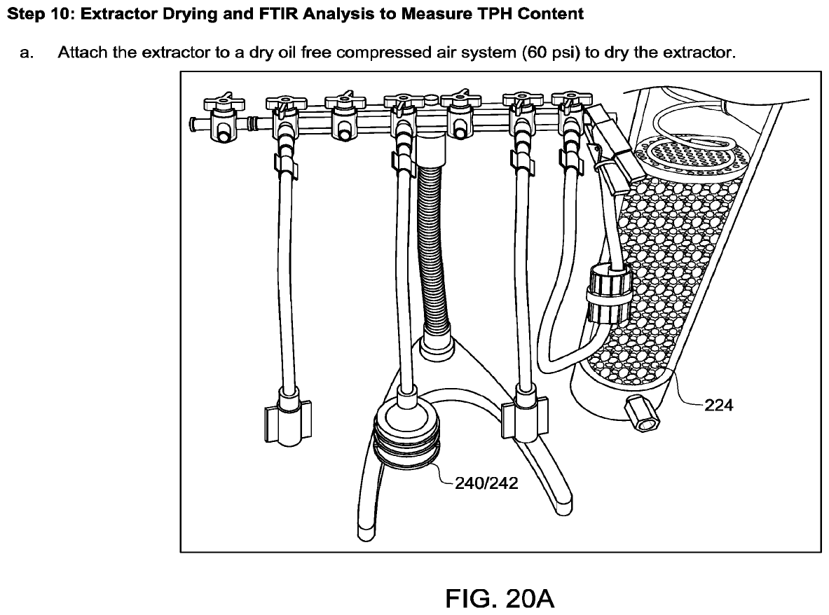

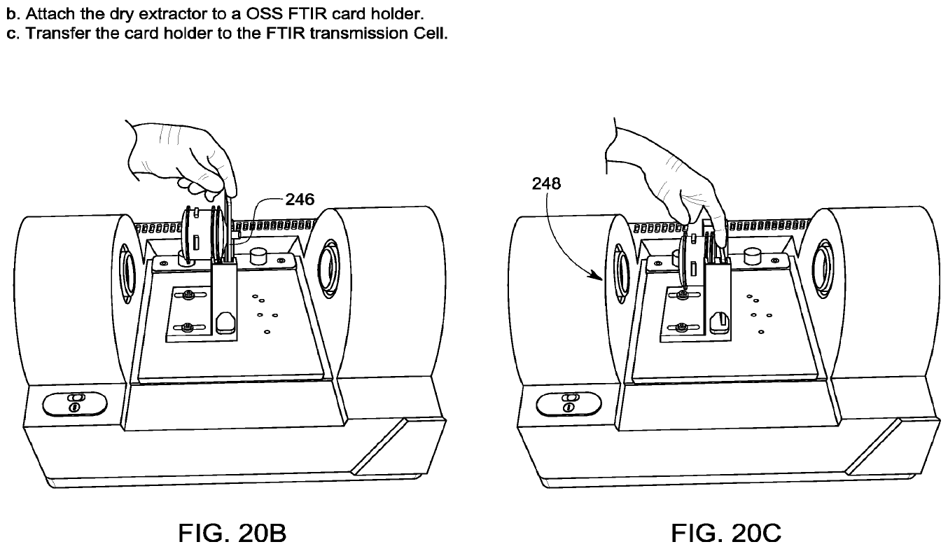

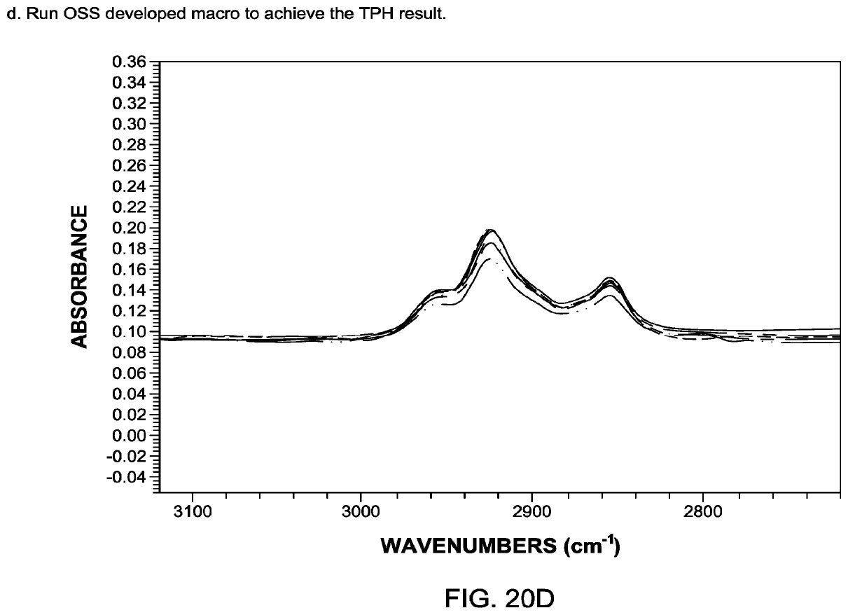

An example of an analysis system that can be used to determine hydrocarbon content of a fluid sample using the membrane 102 as a sample retention device is described in U.S. Pat. No. 8,613,214. The content of that patent is incorporated herein by reference. FIG. 20 also shows steps associated with final sample preparation and FTIR analysis.

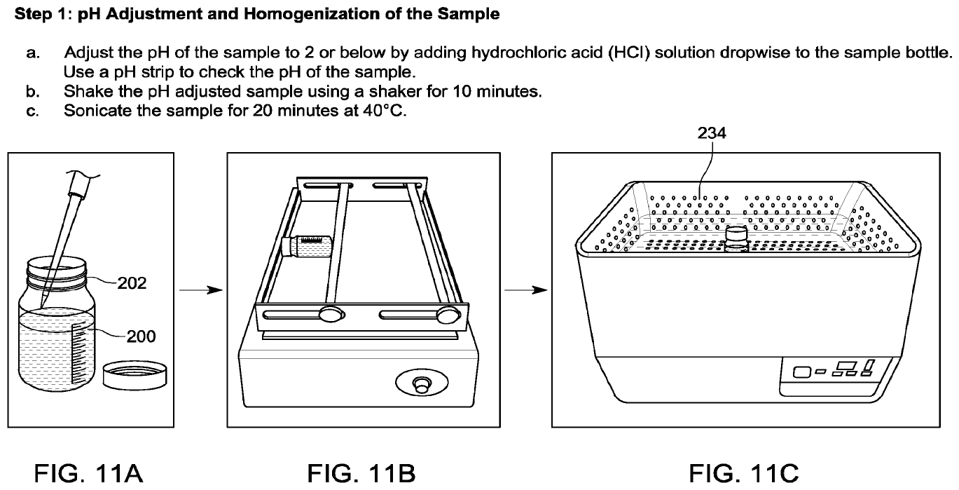

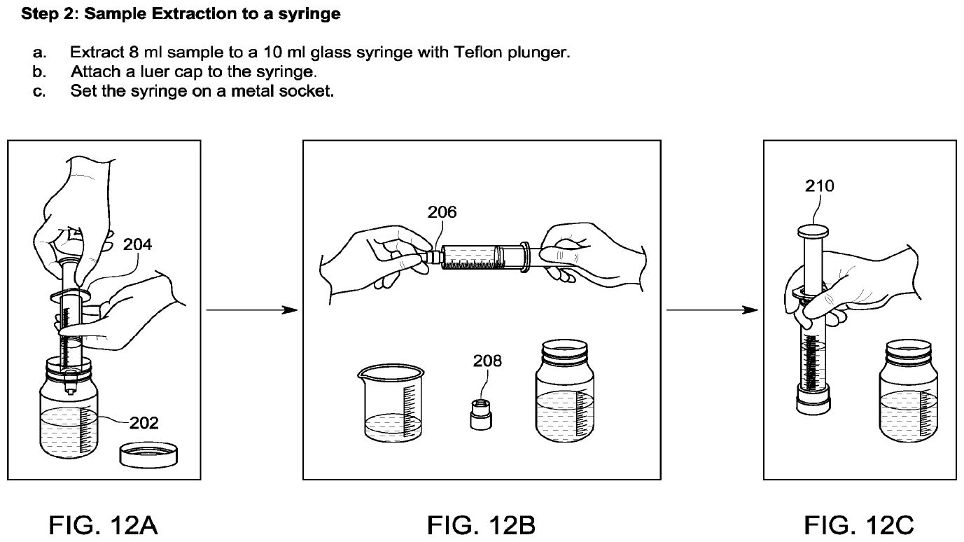

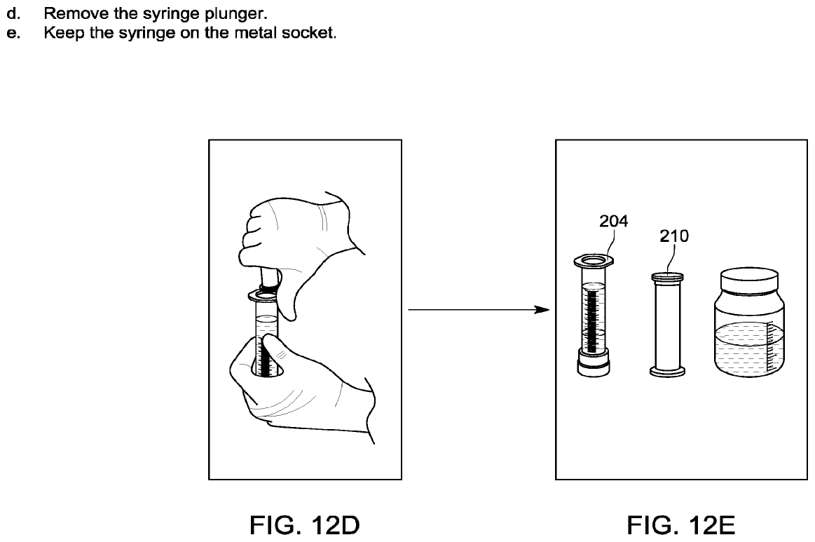

With reference to FIGS. 11A to 11C, which, together with remaining FIGS. 12-19, show and describe steps of a second method for TPH separation by extracting TPH through prior pH adjustment and sample homogenization. A sample 200 in a matrix in container 202 is combined with a pH adjuster. As shown in FIG. 11A, the pH adjuster may be Hydrochloric Acid (HCl) in dropwise fashion until the pH of the solution in the container 202 is at 2 or below. The container 202 is then shaken as shown in FIG. 11B. Next, the container 202 is sonicated in ultrasonic bath 234 for about 20 minutes at about 40 deg C. as shown in FIG. 11C. After sonication, a portion of the sample 200 is removed from the container 202 using a syringe 204 as shown in FIG. 12A. A Luer cap 206 is added to the syringe 204 including the sample portion as shown in FIG. 12B. The syringe 204 is then set on metal socket 208 as shown in FIG. 12C. Plunger 210 of the syringe 204 is removed from the syringe 204 as shown in FIG. 12D. The sample portion is then retained in the syringe 204 with an open end and a closed end contained by the metal socket 208 as shown in FIG. 12E.

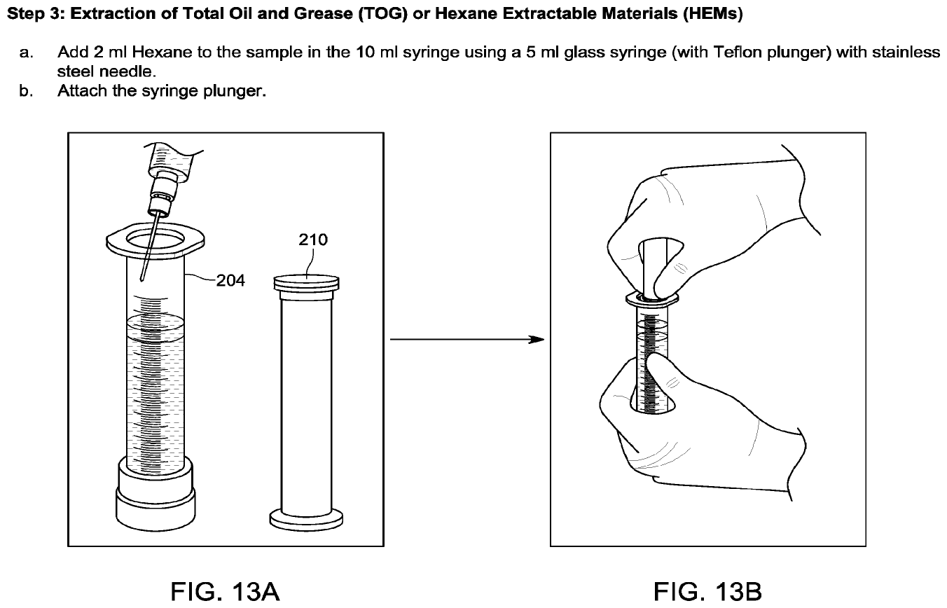

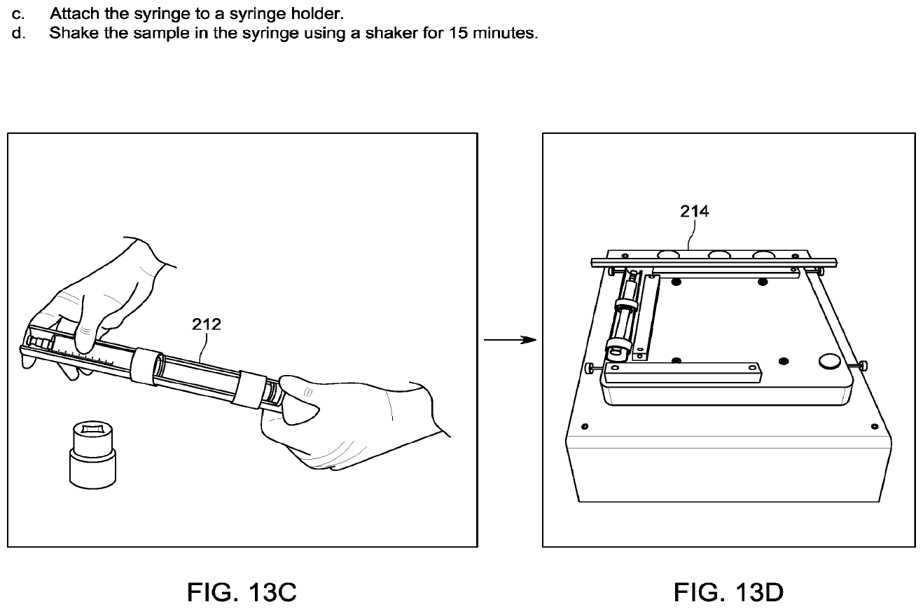

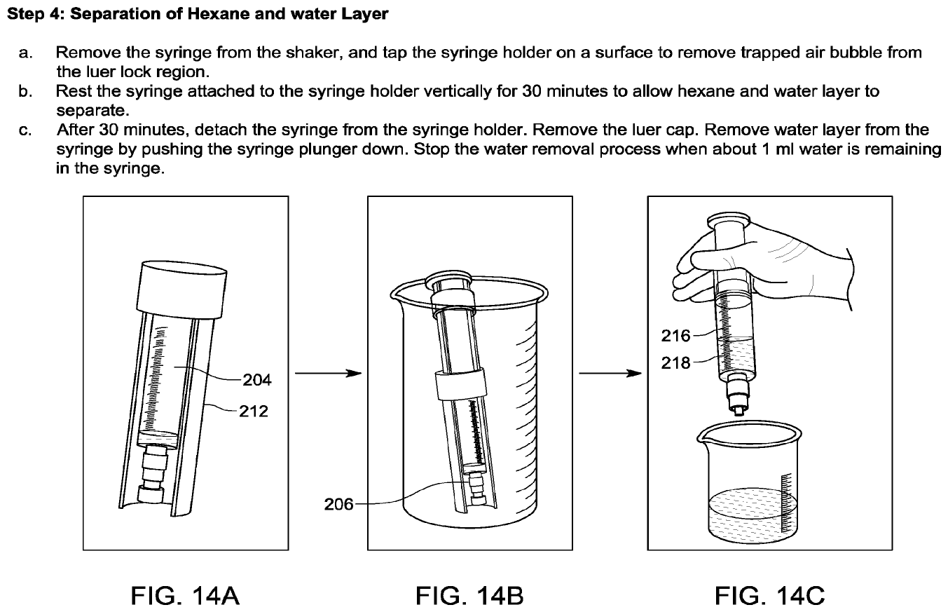

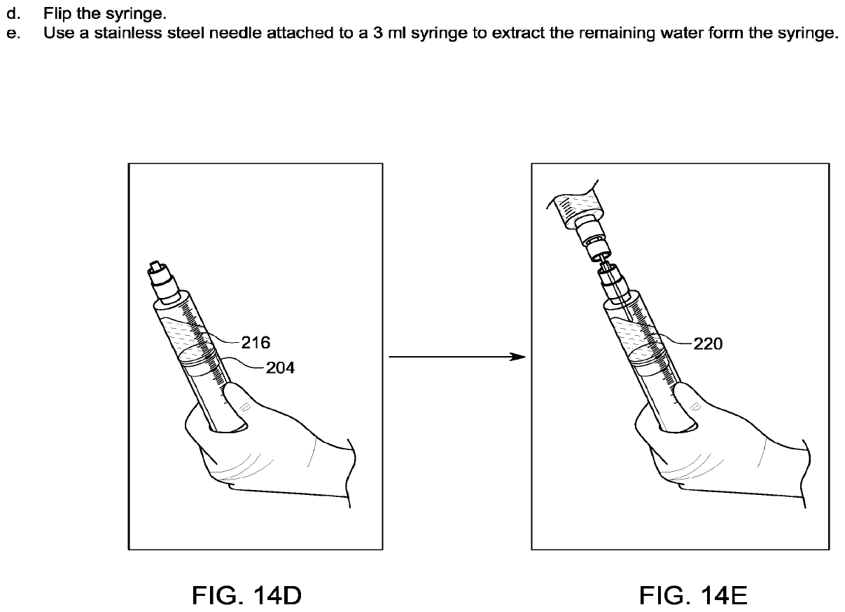

With reference to FIGS. 13 and 14, the sample portion in the syringe 204 is mixed with hexane in the syringe 204 as shown in FIG. 13A. The syringe 204 is then joined with the plunger 210 (FIG. 13B), affixed to syringe holder 212 (FIG. 13C), and that combination is then inserted in shaker 214 (FIG. 13D). The syringe 204 with mixed content is removed from the shaker 214 and tapped to remove trapped air bubbles in the syringe 204 and positioned substantially vertically for a selected period of time, which may be about 30 minutes (FIG. 14A). The contents of the syringe 204 is allowed to separate water and hexane as shown in FIG. 14B by resting in a fixed orientation that is substantially vertical. The syringe 204 is then detached from the holder 212 and the Luer cap 206 is removed. Hexane layer 216 is retained in the syringe 204 while substantially all water 218 is removed by pushing the plunger 210 down to force the water 218 from the syringe 204. Complete water removal is not desired as it would also likely result in removal of a portion of the hexane layer 216. The removal of the water 218 should be halted with about 1 ml of the water layer remaining in the syringe 204 as shown in FIG. 14C. The syringe 204 is then flipped over (FIG. 14D) and a needle 220 that may be a stainless steel needle is attached to the syringe 204 and used to extract the remaining water from within the syringe 204 as shown in FIG. 14E.

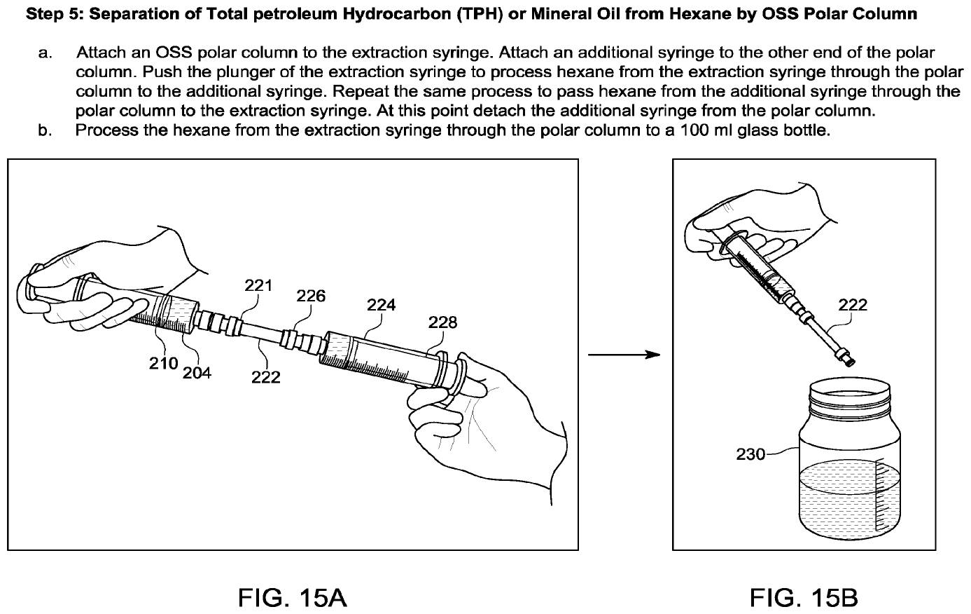

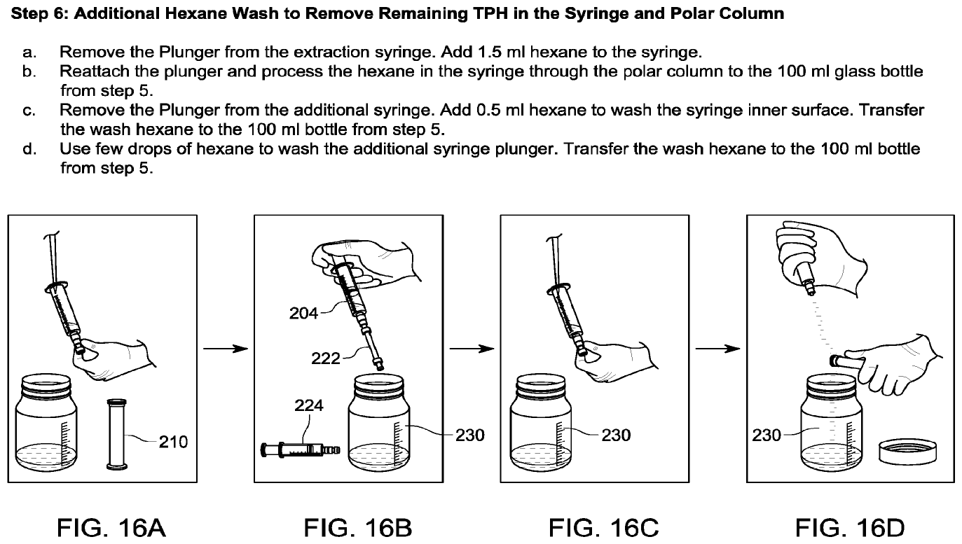

FIG. 15A shows the next step of the process, in which a first end 221 of a polar column 222 is attached to the syringe 204. An additional syringe 224 is attached to a second end 226 of the polar column 222. The plunger 210 of the syringe 204 is actuated to push the hexane layer 216 in the syringe 204 from the syringe 204 through the polar column 222 to the additional syringe 224. A plunger 228 of the additional syringe 224 is then actuated to force the hexane layer 216 back through the polar column 222 to the syringe 204. The additional syringe 224 is then removed from the polar column 222. The hexane layer is then forced from the syringe 204 through the polar column 222 to open container 230, which may be a glass bottle (FIG. 15B). As shown in FIG. 16A, the plunger 210 is removed from the syringe 204 and fresh hexane is added to it. The plunger 210 is then reapplied to the syringe 204, the syringe 204 is reinstalled on the first end 221 of the polar column 222, the additional syringe 224 is reinstalled on the second end 228 of the polar column 222. The process of moving the contents of the syringe 204 back and forth through the polar column 222 is repeated for the fresh hexane (FIG. 16B). The plunger 210 is removed from the syringe 204, a hexane wash is added to the syringe 204 and the contents of the syringe 204 are moved into the container 230 (FIG. 16C). The plunger 210 may be washed with hexane and that wash is transferred into the container 230 (FIG. 16D).

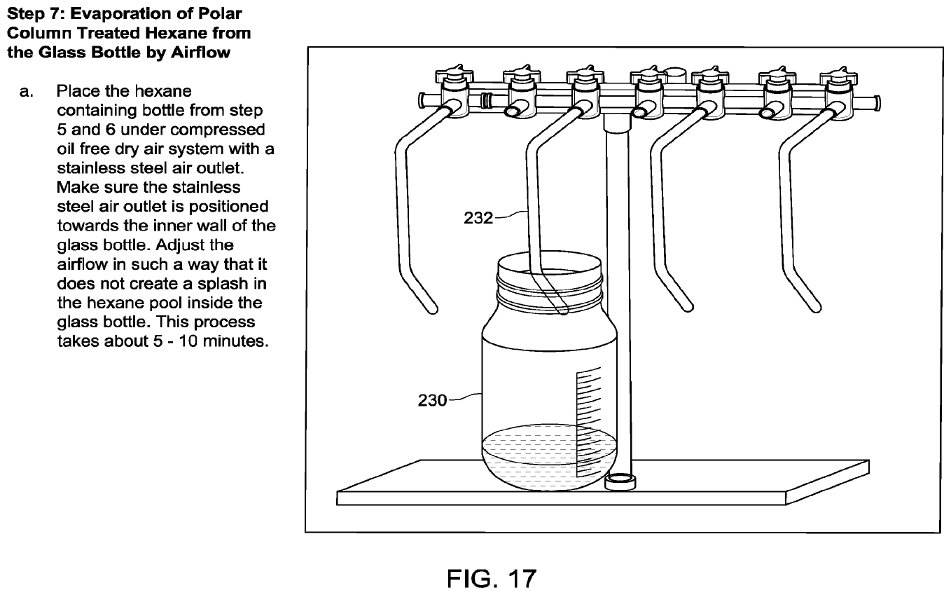

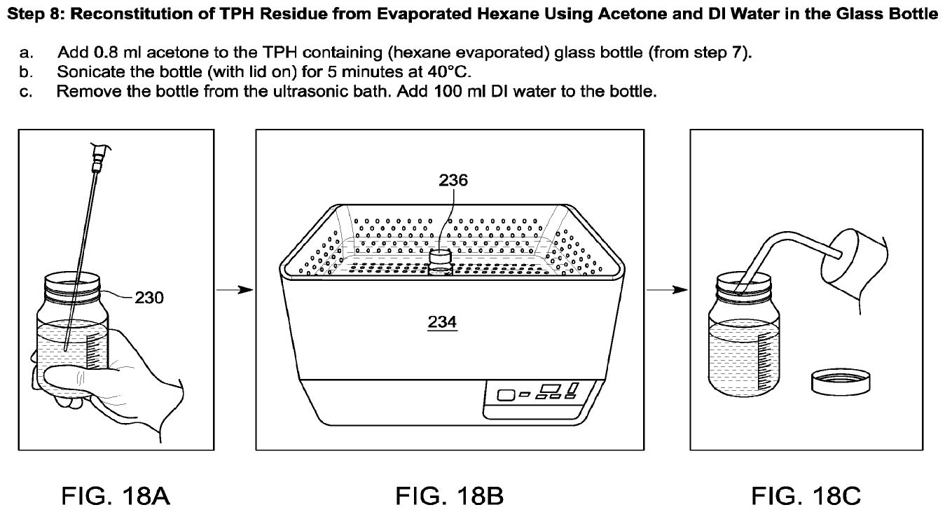

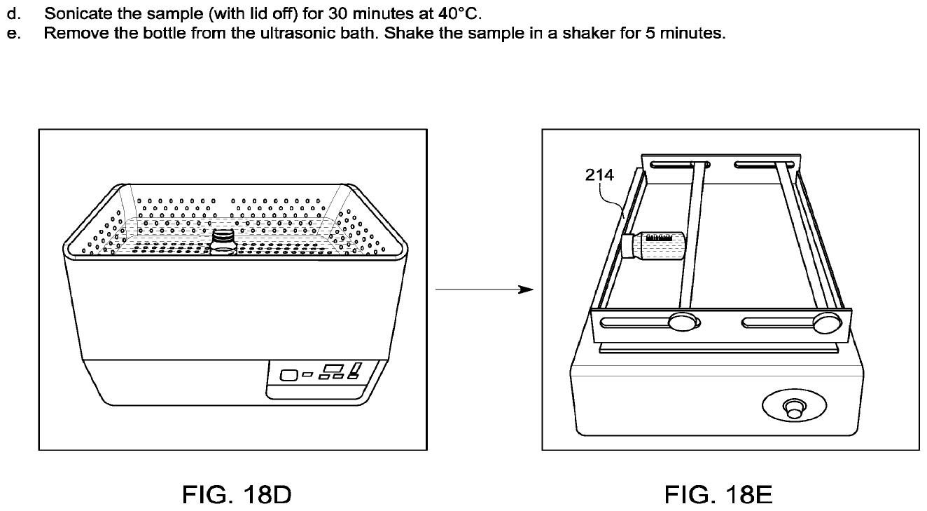

As represented in FIG. 17, the hexane in the container 230 that has passed through the polar column 222 is air dried using an oil-free air drying system having a stainless steel outlet 232. The airflow from the outlet 232 is directed to the contents of the container 203 so as not to create a splash in the container 230. This air drying step evaporates the polar column treated hexane so that just TPH should remain. As shown in FIG. 18, the TPH remaining in the container 230 is reconstituted. Specifically, as shown in FIG. 18A, acetone is added to the container 230. The container 230 is then sonicated in ultrasonic bath 234 with a lid 236 on the container 230 for about five minutes at about 40 deg C. (FIG. 18B). Water is then added to the container 230 after removal from the bath 234 (FIG. 18C). The container 230 is then returned to the bath 234 and sonicated for about 30 minutes at about 40 deg C. with the lid 236 removed as shown in FIG. 18D. The container 230 is then removed from the bath 234 and placed in shaker 214 and shaken for about five minutes (FIG. 18E).

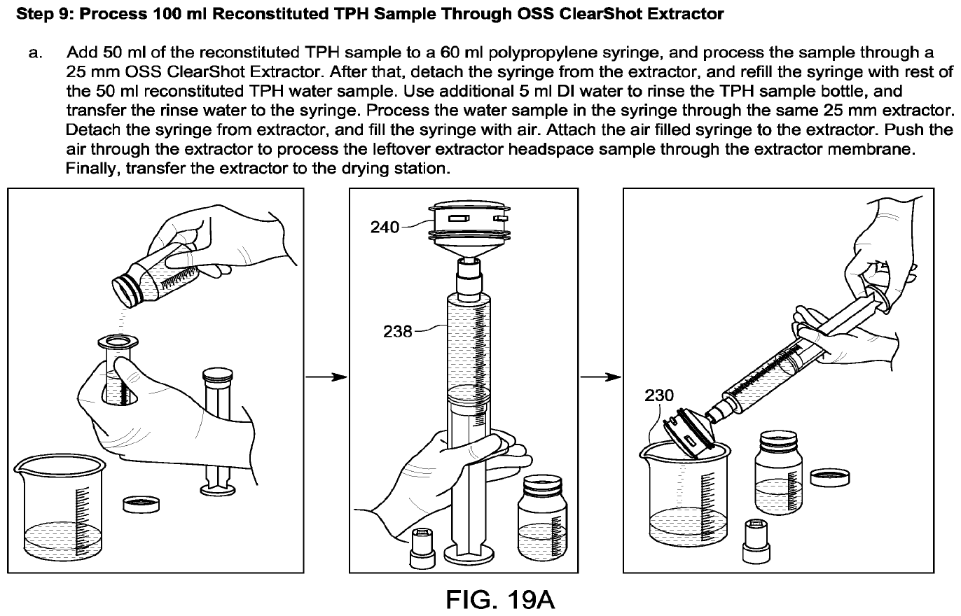

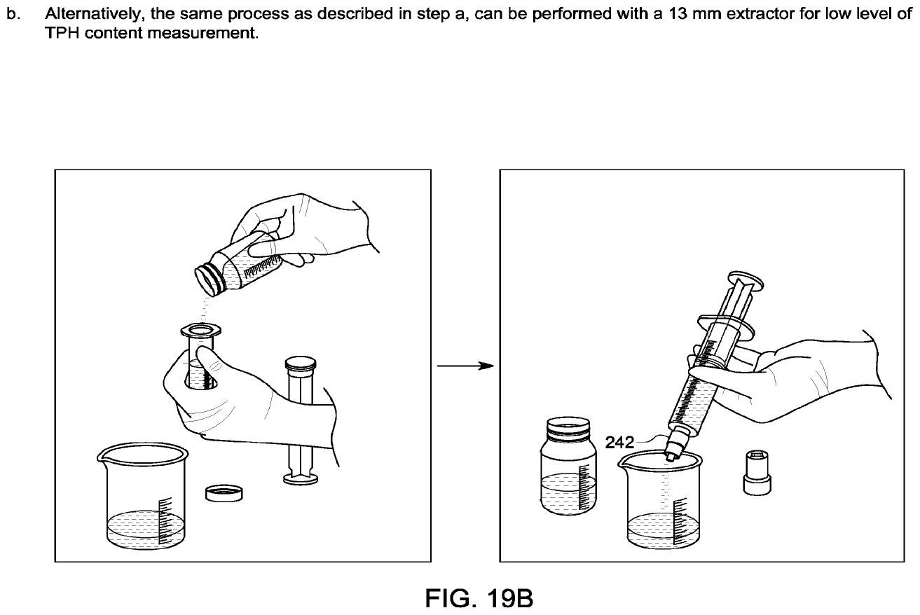

A portion of the contents of the container 230 is added to a TPH syringe 238 which may be a polypropylene syringe as shown in FIG. 19A. It is then processed through a sample extractor such as the 25 mm OSS Clearshot® Extractor 240 shown. The syringe 238 is removed from the Extractor 240 and used to add any remaining solution from the container 230. Water is added to the container 230 and also transferred to the syringe 238. The remaining contents are pass through the Extractor 240. The syringe 238 is removed, filled with air, attached to the Extractor 240, and forced through the Extractor 240. The Extractor 240 is then transferred to a drying station. In an alternative embodiment of the invention shown in FIG. 19B, a 13 mm OSS Clearshot® Extractor 242 may be used to extract and determine low level TPH content measurements relative to what can be determined with Extractor 240.

As shown in FIG. 20A, the selected Extractor 240/242 is dried in an oil-free compressed air system 244. The Extractor 240/242 is then transferred to an analyzer, such as a FTIR card holder 246 but not limited thereto (FIG. 20B). The card holder 246 is then transferred to FTIR transmission cell 248 (FIG. 20C), and the Extractor membrane tested for TPH, with an output such as shown in FIG. 20D the result.

It is to be understood that various modifications of the apparatus 10 as expressly described may be made without departing from the spirit and scope of the invention. Accordingly, other embodiments are within the scope of the claims appended hereto.

What is claimed is:

1. An apparatus for separating hydrocarbons from a matrix sample, wherein the solution sample includes one or more nonpolar hydrocarbons, the apparatus comprising:
a first container arranged to receive the matrix sample;
a second container; and
a connector for coupling the first container and the second container together, wherein the connector is arranged to enable the passage of the matrix sample back and forth between the first container and the second container, wherein the connector includes an interior for capturing a portion of the matrix sample therein while allowing at least a portion of the matrix sample including at least a portion of the one or more nonpolar hydrocarbons to pass therethrough to the second container,
wherein the second container is arranged to transfer the at least portion of the matrix sample to a device configured to enable analysis of the solution sample for detection of the at least one or more nonpolar hydrocarbons.

2. The apparatus as claimed in claim 1, wherein the interior of the connector contains an adsorbent retained by a mesh.

3. The apparatus as claimed in claim 2 wherein the adsorbent is selected from Silica gel and powdered magnesium silicate.

4. The apparatus of claim 1, wherein the first container and the second container are syringes made of glass or any other material that will withstand the contact with materials of the matrix sample.

5. The apparatus of claim 4, wherein each of the first container and the second container includes a plunger arranged to enable repeated transfer of the matrix sample between the first container and the second container.

6. The apparatus of claim 1, wherein the connector is a non-hydrocarbon based and pressured tight tube.

7. A method for separating hydrocarbons from a matrix sample including one or more nonpolar hydrocarbons, the method comprising the steps of:
transferring the matrix sample into a first container;
mixing the sample with a solvent in the first container;
allowing the solvent and the sample to separate into a solvent layer and an aqueous layer with at least a portion of the nonpolar hydrocarbons retained in the solvent layer;
removing the aqueous layer from the first container;
coupling the first container to a connector, wherein the connector is coupled to a second container and the connector includes mans for capturing at least a portion of the solvent layer
passing the solvent layer between the first container and the second container through the connector and permitting at least a portion of the solvent layer including the one or more nonpolar hydrocarbons to pass between the first container and the second container while capturing a portion of the solvent layer containing polar hydrocarbons in the connector; and
transferring the portion of the solvent layer including the one or more nonpolar hydrocarbons to an analyte extraction device.

8. The method of claim 7, further comprising the steps of drying the solvent layer and reconstituting the solvent layer before transferring it to the analyte extraction device.
